(19) United States
(12) Patent Application Publication
(10) Pub. No.: US 2008/0064716 A1
(43) Pub. Date: Mar. 13, 2008

(54) Biphenyl Integrin Antagonists

(75) Inventor: Srinivasan Raj Nagarajan, Chesterfield, MO (US)

Correspondence Address: PFIZER INC, 10555 SCIENCE CENTER DRIVE, SAN DIEGO, CA, 92121, US

(73) Assignee: Pharmacia

(21) Appl. No.: 11/576,584

(22) PCT Filed: Oct. 14, 2004
(86) PCT No.: PCT/US04/33981

§ 371 Date: Apr. 3, 2007

(51) Int. Cl. A61K 31/44; C07D 211/72; C07D 471/02

(52) U.S. Cl. 514/300; 514/352; 546/122; 546/312

(57) ABSTRACT

The following invention is directed to pharmaceutical compounds and compositions of the Formula I 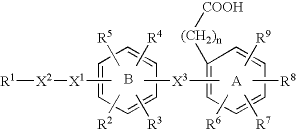
useful for treating conditions mediated by αᵥβ₃ and/or αᵥβ₅ integrin.

### PRIORITY CLAIM TO RELATED PATENT APPLICATION

This patent claims priority to U.S. Provisional Application Ser. No. 60/510,873 (filed Oct. 14, 2003). The entire text of U.S. Provisional Application Ser. No. 60/510,873 is incorporated by reference into this patent.

### FIELD OF THE INVENTION

The following invention is directed to pharmaceutical compounds which are αᵥβ₃ and/or αᵥβ₅ integrin antagonists and as such are useful in pharmaceutical compositions and in methods for treating conditions mediated by αᵥβ₃ and/or αᵥβ₅ integrins.

### BACKGROUND OF THE INVENTION

Antagonists of αᵥβ₃ or dual αᵥβ₃/αᵥβ₅ antagonists can be useful therapeutic agents for treating many pathological conditions, including the treatment or prevention of osteopenia or osteoporosis, or other bone disorders, such as Paget's disease or humoral hypercalcemia of malignancy; neointimal hyperplasia, which can cause artherosclerosis or restenosis after vascular procedures; periodontal disease; treatment and prevention of viral infections or other pathogens; the treatment of neoplasia; pathological angiogenesis or neovascularization such as tumor metastasis, diabetic retinopathy, macular degeneration, rheumatoid arthritis, or osteoarthritis.

Compounds that antagonize the αᵥβ₅ and/or the αᵥβ₃ receptor have been reprinted in the literature.

For example, WO 01/96334 (incorporated by reference in its entirety) provides heteroarylalkanoic acid compounds useful as αᵥβ₃ and/or αᵥβ₅ inhibitors.

WO 97/08145 provides meta-gaunidine, urea, thiourea or azacyclic amino benzoic acid compounds and derivatives useful as αᵥβ₃ and/or αᵥβ₅ inhibitors.

WO 97/36859 provides para-substituted phenylene derivatives useful as αᵥβ₃ and/or αᵥβ₅ inhibitors.

WO 97/36861 provides meta-substituted sulphonamide phenylene derivatives useful as αᵥβ₃ and/or αᵥβ₅ inhibitors.

WO 97/36860 provides cinnamic acid derivatives useful as αᵥβ₃ and/or αᵥβ₅ inhibitors.

WO 97/36858 provides cyclopropyl alkanoic acid derivatives useful as αᵥβ₃ and/or αᵥβ₅ inhibitors.

WO 97/36862 provides meta-substituted phenylene derivatives useful as αᵥβ₃ and/or αᵥβ₅ inhibitors.

WO 99/52896 provides heterocyclic glycyl-beta alanine derivatives useful as αᵥβ₃ and/or αᵥβ₅ inhibitors.

WO 00/51968 provides meta-azacyclic amino benzoic acid compounds and derivatives useful as αᵥβ₃ and/or αᵥβ₅ inhibitors.

WO 01/96310 provides dihydrostilbene alkanoic acid derivatives useful as αᵥβ₃ and/or αᵥβ₅ inhibitors.

WO 02/18340 provides cycloalkyl compounds useful as αᵥβ₃ and/or αᵥβ₅ inhibitors.

WO 02/18377 provides bicyclic compounds useful as αᵥβ₃ and/or αᵥβ₅ inhibitors.

WO 02/26717 provides hydroxy acid compounds useful as αᵥβ₃ and/or αᵥβ₅ inhibitors.

WO 02/26227 provides lactone compounds useful as αᵥβ₃ and/or αᵥβ₅ inhibitors.

### SUMMARY OF THE INVENTION

As evidenced by the continuing research in integrin antagonists and by the shortcomings of the compounds and methods of the art, there still remains a need for small-molecule, non-peptidic selective αᵥβ₃ and/or αᵥβ₅ antagonists that display decreased side-effects, improved potency, pharmacodynamic and pharmacokinetic properties, such as oral bioavailability and duration of action, over already described compounds. Such compounds would prove to be useful for the treatment, prevention, or suppression of various pathologies enumerated above that are mediated by αᵥβ₃ and/or αᵥβ₅ receptor binding and cell adhesion and activation.

In one embodiment, the present invention comprises a class of biphenyl integrin antagonists.

The present invention relates to a class of compounds represented by the Formula I: 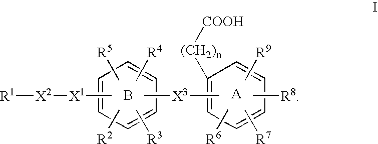
or a pharmaceutically acceptable salt, ester or tautomer thereof, wherein:

A and B are phenyl;

n is an integer from 1 to 3;

X¹ is selected from O, NR, S, SO, SO₂, CHR and CH₂, wherein:
R is selected from the group consisting of hydrogen, aryl, and heterocyclyl;

X² is selected from the group consisting of alkyl, alkylamino, aminoalkyl, alkylaminoalkyl, alklthio, thioalkyl, alkylthioalkyl, alkylsulfonyl, sulfonylalkyl, alkylsulfonylalkyl, oxyalkyl, alkoxyalkyl, and alkoxy;

X³ is C₁-C₆ alkyl;

R¹ is selected from the group consisting of pyridinyl and napthyridinyl, wherein:
either is optionally substituted with a substituent selected from the group consisting of hydrogen, alkyl, halo, and amino;

R², R³R⁴, R⁵, R⁶, R⁷, R⁸ and R⁹ are independently selected from the group consisting of hydrogen, alkyl, alkenyl, alkoxy, alkoxyalkyl, alkoxycarbonylalkyl, alkylamino, alkylcarbonyl, alkylheteroaryl, alkylsulfonylalkyl, alkylthio, alkynyl, aminocarbonylalkyl, cyano, dialkylamino, halo, haloalkoxy, haloalkyl, hydroxy and hydroxyalkyl; and

X³ is independently meta- or para- to the X¹ of the B ring, and wherein further X³ is ortho-, meta-, or para- to the carboxylic acid chain of the A ring.

In another embodiment, the invention comprises pharmaceutical compositions comprising compounds of the Formula I. Such compounds and compositions are useful in selectively inhibiting or antagonizing the αᵥβ₃ and/or αᵥβ₅ integrins and therefore in another embodiment the present invention relates to a method of selectively inhibiting or antagonizing the αᵥβ₃ and/or αᵥβ₅ integrin.

In another embodiment, the invention provides methods of treating or inhibiting pathological conditions associated therewith such as osteoporosis, humoral hypercalcemia of malignancy, Paget's disease, tumor metastasis, solid tumor growth (neoplasia), angiogenesis, including tumor angiogenesis, retinopathy including macular degeneration and diabetic retinopathy, arthritis including rheumatoid arthritis and osteoarthritis, periodontal disease, psoriasis, smooth muscle cell migration and restenosis in a mammal in need of such treatment. Additionally, such pharmaceutical agents are useful as antiviral agents and antimicrobials. The compounds of the present invention may be used alone or in combination with other pharmaceutical agents.

### DETAILED DESCRIPTION

The compounds of this invention include 1) αᵥβ₃ integrin antagonists; or 2) αᵥβ₅ integrin antagonists; or 3) mixed or dual αᵥβ₃/αᵥβ₅ antagonists. The present invention includes compounds which inhibit the respective integrins and also includes pharmaceutical compositions comprising such compounds. The present invention further provides for methods for treating or preventing conditions mediated by the αᵥβ₃ and/or αᵥβ₅ receptors in a mammal in need of such treatment comprising administering a therapeutically effective amount of the compounds of the present invention and pharmaceutical compositions of the present invention. Administration of such compounds and compositions of the present invention inhibits angiogenesis, tumor metastasis, tumor growth, skeletal malignancy of breast cancer, osteoporosis, Paget's disease, humoral hypercalcemia of malignancy, retinopathy, macular degeneration, arthritis including rheumatoid arthritis and osteoarthritis, periodontal disease, smooth muscle cell migration, including restenosis and artherosclerosis, and microbial or viral diseases. The compounds of the present invention can be used, alone or in combination with other therapeutic agents, in the treatment or modulation of various conditions or disease states including tumor metastasis, solid tumor growth (neoplasia), osteoporosis, Paget's disease, humoral hypercalcemia of malignancy, osteopenia, endometriosis, angiogenesis, including tumor angiogenesis, retinopathy including macular degeneration, arthritis, including rheumatoid arthritis and osteoarthritis, periodontal disease, psoriasis and smooth muscle cell migration (e.g. restenosis and artherosclerosis. Additionally, it has been found that such agents would be useful as antivirals, antifungals and antimicrobials. Thus, compounds which selectively antagonize αᵥβ₃ would be beneficial for treating such conditions.

In order to prevent bleeding side effects associated with the inhibition of α_{IIb}β₃, it would be beneficial to have a high selectivity ratio of αᵥβ₃ and αᵥβ₅ over α_{IIb}β₃. The compounds of the present invention include selective antagonists of αᵥβ₃ over α_{IIb}β₃.

The compounds of the present invention further show greater selectivity for the αᵥβ₃ and/or αᵥβ₅ integrin than for the αᵥβ₆ integrin. It has been found that the selective antagonism of the αᵥβ₃ integrin is desirable in that the αᵥβ₆ integrin may play a role in normal physiological processes of tissue repair and cellular turnover that routinely occur in the skin and pulmonary tissue, and the inhibition of this function can be deleterious (Huang et al., Am J Respir Cell Mol Biol 1998, 19(4): 636-42). Therefore, compounds of the present invention which selectively inhibit the αᵥβ₃ integrin as opposed to the αᵥβ₆ integrin have reduced side effects associated with inhibition of the αᵥβ₆ integrin.

Compounds

The present invention comprises a class of biphenyl integrin antagonists.

In one embodiment, the present invention relates to a class of compounds represented by the formula I: 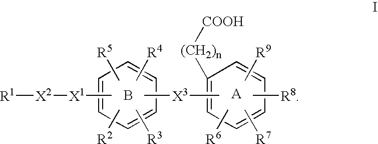
or a pharmaceutically acceptable salt, ester or tautomer thereof, wherein:

A and B are phenyl;

n is an integer from 1 to 3;

X¹ is selected from the group consisting of O, NR, S, SO, SO₂, CHR and CH₂, wherein;
R is selected from the group consisting of hydrogen, aryl, and heterocyclyl;

X² is selected from the group consisting of alkyl, alkylamino, aminoalkyl, alkylaminoalkyl, alklthio, thioalkyl, alkylthioalkyl, alkylsulfonyl, sulfonylalkyl, alkylsulfonylalkyl, oxyalkyl, alkoxyalkyl, and alkoxy;

X³ is C₁-C₆ alkyl;

R¹ is selected from the group consisting of pyridinyl and napthyridinyl, wherein either can be optionally substituted with a substituent selected from the group consisting of hydrogen, alkyl, halo, and amino;

R², R³R⁴, R⁵, R⁶, R⁷, R⁸ and R⁹ are independently selected from the group consisting of hydrogen, alkyl, alkenyl, alkoxy, alkoxyalkyl, alkoxycarbonylalkyl, alkylamino, alkylcarbonyl, alkylheteroaryl, alkylsulfonylalkyl, alkylthio, alkynyl, aminocarbonylalkyl, cyano, dialkylamino, halo, haloalkoxy, haloalkyl, hydroxy and hydroxyalkyl; and

X³ is independently meta- or para- to the X¹ of the B ring, and X³ is further ortho-, meta-, or para- to the carboxylic acid chain of the A ring.

In another embodiment, the present invention consists of those compounds of formula I, wherein:

n is an integer from 1 to 2;

X¹ is selected from the group consisting of O, NH, and CH₂;

X² is C₁-C₆ alkyl or C₁-C₆ alkylamino;

X³ is —CH₂—;

R¹ is selected from the group consisting of pyridinyl, pyridinylamino and napthyridinyl; and

R², R³, R⁴, R⁶, R⁷, R⁸ and R⁹ are independently selected from hydrogen, C₁-C₆ alkyl, alkenyl, alkynyl, alkoxy, halo, haloalkyl and hydroxy.

In another embodiment, the present invention consists of those compounds of formula I, wherein:

n is an integer from 1 to 2;

X¹ is O;

X² is C₁-C₆ alkyl or C₁-C₆ alkylamino;

X³ is —CH₂—;

R¹ is unsubstituted pyridinyl or napthyridinyl; and

R², R³, R⁴, R⁵, R⁶, R⁷, R⁸ and R⁹ are each hydrogen.

In yet another embodiment, X² is ethyl or propyl.

In another embodiment of the present invention, the compounds of the present invention have the structure of formula II: 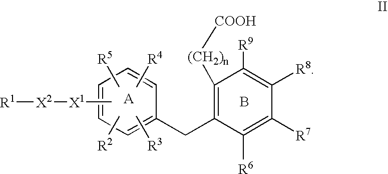
or a pharmaceutically acceptable salt, ester or tautomer thereof, wherein:

A and B are phenyl;

n is an integer from 1 to 3;

R is selected from the group consisting of hydrogen, aryl, and heterocyclyl;
X² is selected from the group consisting of alkyl, alkylamino, aminoalkyl, alkylaminoalkyl, alklthio, thioalkyl, alkylthioalkyl, alkylsulfonyl, sulfonylalkyl, alkylsulfonylalkyl, oxyalkyl, alkoxyalkyl, and alkoxy;

R¹ is selected from the group consisting of pyridinyl and napthyridinyl;

R², R³R⁴, R⁵, R⁶, R⁷, R⁸ and R⁹ are independently selected from hydrogen, alkyl, alkenyl, alkoxy, alkoxyalkyl, alkoxycarbonylalkyl, alkylamino, alkylcarbonyl, alkylheteroaryl, alkylsulfonylalkyl, alkylthio, alkynyl, aminocarbonylalkyl, cyano, dialkylamino, halo, haloalkoxy, haloalkyl, hydroxy and hydroxyalkyl.

In another embodiment, the present invention consists of those compounds of formula II, wherein:

n is an integer from 1 to 2;

X¹ is selected from O, NH, or CH₂;

X² is C₁-C₆ alkyl or C₁-C₆ alkylamino;

R¹ is pyridinyl or napthyridinyl; and

R², R³, R⁴, R⁶, R⁷, R⁸ and R⁹ are independently selected from hydrogen, C₁-C₆ alkyl, alkenyl, alkynyl, alkoxy, halo, haloalkyl and hydroxy.

In another embodiment, the present invention consists of those compounds of formula II, wherein;

n is an integer from 1 to 2;

X¹ is O;

X² is C₁-C₆ alkyl or C₁-C₆ alkylamino;

R¹ is pyridinyl or napthyridinyl; and

R², R³, R⁴, R⁵, R⁶, R⁷, R⁸ and R⁹ are each hydrogen.

In yet another embodiment, X² is ethyl or propyl.

In another embodiment of the present invention, the present invention relates to a class of compounds represented by the formula III:

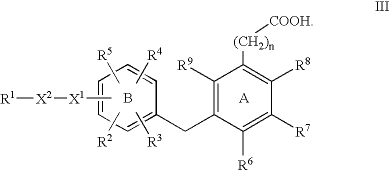

or a pharmaceutically acceptable salt, ester or tautomer thereof,

wherein:

A and B are phenyl;

n is an integer from 1 to 3;

X¹ is selected from O, NR, S, SO, SO₂, CHR and CH₂, wherein;
R is selected from the group consisting of hydrogen, aryl, and heterocyclyl;

X² is selected from the group consisting of alkyl, alkylamino, aminoalkyl, alkylaminoalkyl, alklthio, thioalkyl, alkylthioalkyl, alkylsulfonyl, sulfonylalkyl, alkylsulfonylalkyl, oxyalkyl, alkoxyalkyl, and alkoxy;

R¹ is selected from the group consisting of pyridinyl and napthyridinyl;

R², R³, R⁴, R⁵, R⁶, R⁷, R⁸ and R⁹ are independently selected from hydrogen, alkyl, alkenyl, alkoxy, alkoxyalkyl, alkoxycarbonylalkyl, alkylamino, alkylcarbonyl, alkylheteroaryl, alkylsulfonylalkyl, alkylthio, alkynyl, aminocarbonylalkyl, cyano, dialkylamino, halo, haloalkoxy, haloalkyl, hydroxy and hydroxyalkyl; and

X¹ of ring B is attached meta- or para- to the methylene bridge attaching rings

A and B.

In another embodiment, the present invention consists of those compounds of formula III, wherein:

n is an integer from 1 to 2;

X¹ is selected from O, NH, or CH₂;

X² is C₁-C₆ alkyl or C₁-C₆ alkylamino;

R¹ is pyridinyl or napthyridinyl; and

R², R³, R⁴, R⁶, R⁷, R⁸ and R⁹ are independently selected from hydrogen, C₁-C₆ alkyl, alkenyl, alkynyl, alkoxy, halo, haloalkyl and hydroxy.

In another embodiment, the present invention consists of those compounds of formula III, wherein:

n is an integer from 1 to 2;

X¹ is O;

X² is C₁-C₆ alkyl or C₁-C₆ alkylamino;

R¹ is pyridinyl or napthyridinyl; and

R², R³, R⁴, R⁵, R⁶, R⁷, R⁸ and R⁹ are each hydrogen. In yet another embodiment, X² is ethyl or propyl.

In another embodiment of the present invention, the present invention relates to a class of compounds represented by the formula IV: 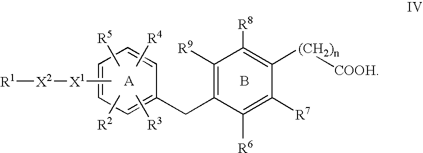
or a pharmaceutically acceptable salt, ester or tautomer thereof, wherein:
n is an integer from 1 to 3;
X¹ is selected from O, NR, S, SO, SO₂, CHR and CH₂, wherein:
   R is selected from the group consisting of hydrogen, aryl, and heterocyclyl;
X² is selected from the group consisting of alkyl, alkylamino, aminoalkyl, alkylaminoalkyl, alklthio, thioalkyl, alkylthioalkyl, alkylsulfonyl, sulfonylalkyl, alkylsulfonylalkyl, oxyalkyl, alkoxyalkyl, and alkoxy;
R¹ is selected from the group consisting of pyridinyl and napthyridinyl; and

R², R³R⁴, R⁵, R⁶, R⁷, R⁸ and R⁹ are independently selected from the group consisting of hydrogen, alkyl, alkenyl, alkoxy, alkoxyalkyl, alkoxycarbonylalkyl, alkylamino, alkylcarbonyl, alkylheteroaryl, alkylsulfonylalkyl, alkylthio, alkynyl, aminocarbonylalkyl, cyano, dialkylamino, halo, haloalkoxy, haloalkyl, hydroxy and hydroxyalkyl.

In another embodiment, the present invention consists of those compounds of formula IV, wherein:

n is an integer from 1 to 2;

X¹ is selected from O, NH, or CH₂;

X² is C₁-C₆ alkyl or C₁-C₆ alkylamino;

R¹ is pyridinyl or napthyridinyl; and

R², R³, R⁴, R⁶, R⁷, R⁸ and R⁹ are independently selected from hydrogen, C₁-C₆ alkyl, alkenyl, alkynyl, alkoxy, halo, haloalkyl and hydroxy.

In another embodiment, the present invention consists of those compounds of formula IV, wherein:

n is an integer from 1 to 2;

X¹ is O;

X² is C₁-C₆ alkyl or C₁-C₆ alkylamino;

R¹ is pyridinyl or napthyridinyl; and

R², R³, R⁴, R⁵, R⁶, R⁷, R⁸ and R⁹ are each hydrogen.

In yet another embodiment, X² is ethyl or propyl.

In another embodiment of the present invention, the present invention relates to a class of compounds represented by the formula V: 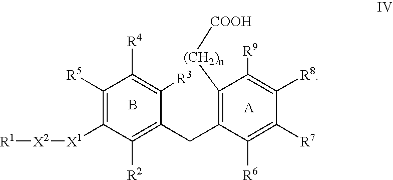
or a pharmaceutically acceptable salt, ester or tautomer thereof, wherein:

n is an integer from 1 to 3;

X¹ is selected from O, NR, S, SO, SO₂, CHR and CH₂, wherein;
R is selected from the group consisting of hydrogen, aryl, and heterocyclyl; X² is selected from the group consisting of alkyl, alkylamino, aminoalkyl, alkylaminoalkyl, alklthio, thioalkyl, alkylthioalkyl, alkylsulfonyl, sulfonylalkyl, alkylsulfonylalkyl, oxyalkyl, alkoxyalkyl, and alkoxy;

R¹ is selected from the group consisting of pyridinyl and napthyridinyl; and

R², R³R⁴, R⁵, R⁶, R⁷, R⁸ and R⁹ are independently selected from the group consisting of hydrogen, alkyl, alkenyl, alkoxy, alkoxyalkyl, alkoxycarbonylalkyl, alkylamino, alkylcarbonyl, alkylheteroaryl, alkylsulfonylalkyl, alkylthio, alkynyl, aminocarbonylalkyl, cyano, dialkylamino, halo, haloalkoxy, haloalkyl, hydroxy and hydroxyalkyl.

In another embodiment, the present invention consists of those compounds of formula V, wherein:

n is an integer from 1 to 2;

X¹ is selected from O, NH, or CH₂;

X² is C₁-C₆ alkyl or C₁-C₆ alkylamino;

R¹ is pyridinyl or napthyridinyl; and

R², R³, R⁴, R⁶, R⁷, R⁸ and R⁹ are independently selected from hydrogen, C₁-C₆ alkyl, alkenyl, alkynyl, alkoxy, halo, haloalkyl and hydroxy.

In another embodiment, the present invention consists of those compounds of formula V, wherein:

n is an integer from 1 to 2;

X¹ is O;

X² is C₁-C₆ alkyl or C₁-C₆ alkylamino;

R¹ is pyridinyl or napthyridinyl; and

R², R³, R⁴, R⁵, R⁶, R⁷, R⁸ and R⁹ are each hydrogen.

In yet another embodiment, X² is ethyl or propyl.

In another embodiment of the present invention, the present invention relates to a class of compounds represented by the formula VI: 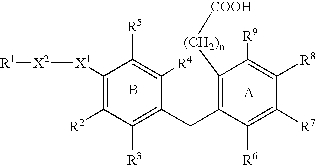
or a pharmaceutically acceptable salt, ester or tautomer thereof, wherein:

A and B are phenyl;

n is an integer from 1 to 3;

X¹ is selected from O, NR, S, SO, SO₂, CHR and CH₂, wherein;
R is selected from the group consisting of hydrogen, aryl, and heterocyclyl;

X² is selected from the group consisting of alkyl, alkylamino, aminoalkyl, alkylaminoalkyl, alklthio, thioalkyl, alkylthioalkyl, alkylsulfonyl, sulfonylalkyl, alkylsulfonylalkyl, oxyalkyl, alkoxyalkyl, and alkoxy;

R¹ is selected from the group consisting of pyridinyl and napthyridinyl;

R², R³R⁴, R⁵, R⁶, R⁷, R⁸ and R⁹ are independently selected from hydrogen, alkyl, alkenyl, alkoxy, alkoxyalkyl, alkoxycarbonylalkyl, alkylamino, alkylcarbonyl, alkylheteroaryl, alkylsulfonylalkyl, alkylthio, alkynyl, aminocarbonylalkyl, cyano, dialkylamino, halo, haloalkoxy, haloalkyl, hydroxy and hydroxyalkyl; and

X¹ of ring B is attached meta- or para- to the methylene bridge;

In another embodiment, the present invention consists of those compounds of formula VI, wherein;

n is an integer from 1 to 2;

X¹ is selected from O, NH, or CH₂;

X² is C₁-C₆ alkyl or C₁-C₆ alkylamino;

R¹ is pyridinyl or napthyridinyl; and

R², R³, R⁴, R⁶, R⁷, R⁸ and R⁹ are independently selected from hydrogen, C₁-C₆ alkyl, alkenyl, alkynyl, alkoxy, halo, haloalkyl and hydroxy.

In another embodiment, the present invention consists of those compounds of formula VI, wherein:

n is an integer from 1 to 2;

X¹ is O;

X² is C₁-C₆ alkyl or C₁-C₆ alkylamino;

R¹ is pyridinyl or napthyridinyl; and

R², R³, R⁴, R⁵, R⁶, R⁷, R⁸ and R⁹ are each hydrogen.

In yet another embodiment, X² is ethyl or propyl.

In another embodiment of the present invention, the compounds of the present invention have the structure of formula VII: 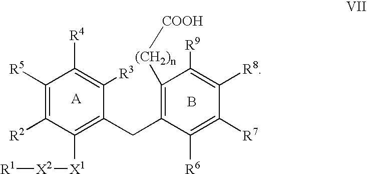
or a pharmaceutically acceptable salt, ester or tautomer thereof, wherein:

A and B are phenyl;

n is an integer from 1 to 3;

R is selected from the group consisting of hydrogen, aryl, and heterocyclyl;
X² is selected from the group consisting of alkyl, alkylamino, aminoalkyl, alkylaminoalkyl, alklthio, thioalkyl, alkylthioalkyl, alkylsulfonyl, sulfonylalkyl, alkylsulfonylalkyl, oxyalkyl, alkoxyalkyl, and alkoxy;

R¹ is selected from the group consisting of pyridinyl and napthyridinyl;

R², R³, R⁴, R⁵, R⁶, R⁷, R⁸ and R⁹ are independently selected from hydrogen, alkyl, alkenyl, alkoxy, alkoxyalkyl, alkoxycarbonylalkyl, alkylamino, alkylcarbonyl, alkylheteroaryl, alkylsulfonylalkyl, alkylthio, alkynyl, aminocarbonylalkyl, cyano, dialkylamino, halo, haloalkoxy, haloalkyl, hydroxy and hydroxyalkyl.

In another embodiment, the present invention consists of those compounds of formula VII, wherein:

n is an integer from 1 to 2;

X¹ is selected from O, NH, or CH₂;

X² is C₁-C₆ alkyl or C₁-C₆ alkylamino;

R¹ is pyridinyl or napthyridinyl; and

R², R³, R⁴, R⁶, R⁷, R⁸ and R⁹ are independently selected from hydrogen, C₁-C₆ alkyl, alkenyl, alkynyl, alkoxy, halo, haloalkyl and hydroxy.

In another embodiment, the present invention consists of those compounds of formula VII, wherein:

n is an integer from 1 to 2;

X¹ is O;

X² is C₁-C₆ alkyl or C₁-C₆ alkylamino;

R¹ is pyridinyl or napthyridinyl; and

R², R³, R⁴, R⁵, R⁶, R⁷, R⁸ and R⁹ are each hydrogen.

In yet another embodiment, X² is ethyl or propyl.

The compounds of the present invention can have additional chiral centers and occur as diastereomeric mixtures, and as isomers as defined above.

The compounds as shown above can exist in various isomeric forms. For example, the carbon of the beta amino acid may be in the R- or S-positions. As used herein, the term “isomer” refers to all isomers except enantiomers. Tautomeric forms are also included as well as pharmaceutically acceptable salts of such isomers and tautomers.

In the structures and formulas herein, a bond drawn across a bond of a ring can be to any available atom on the ring.

The term “pharmaceutically acceptable salt” refers to a salt prepared by combining a compound of Formula I-VII with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid. Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. In a further embodiment, representative salts include the following: benzenesulfonate, hydrobromide and hydrochloride. In one embodiment, the chloride salt is useful for medical purposes. Furthermore, where the compounds of the invention carry an acidic moiety, suitable pharmaceutically acceptable salts thereof may include alkali metal salts, e.g., sodium or potassium salts; alkaline earth metal salts, e.g., sodium, potassium, calcium or magnesium salts; and salts formed with suitable organic ligands, e.g., quaternary ammonium salts.

All of the pharmacologically acceptable salts may be prepared by conventional means. (See Berge et al., J Pharm. Sci., 1977, 66(1): 1-19 for additional examples of pharmaceutically acceptable salts, which is incorporated by reference herein in its entirety.)

Also included within the scope of the invention are polymorphs, or hydrates or other modifiers of the compounds of invention.

The present invention includes within its scope prodrugs of the compounds of this invention. In general, such prodrugs will be functional derivatives of the compounds of this invention which are readily convertible in vivo into the required compound. For example, prodrugs of a carboxylic acid may include an ester, an amide, or an ortho-ester. Thus, in the methods of treatment of the present invention, the term “administering” shall encompass the treatment of the various conditions described with the compound specifically disclosed or with a compound which may not be specifically disclosed, but which converts to the compound of Formula I in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

### DEFINITIONS

The following is a list of definitions of various terms used herein:

As used herein the term “acyl” refers to a radical of the formula 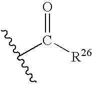
wherein R²⁶ is alkyl, alkenyl, and alkynyl, all optionally substituted thereon as defined herein. Encompassed by such radical are the groups acetyl and the like.

As used herein the term “alkenyl” refers to unsaturated acyclic hydrocarbon radicals containing at least one double bond and 2 to about 6 carbon atoms, more preferably from 2 to about 3 carbon atoms, which carbon-carbon double bond may have either cis or trans geometry within the alkenyl moiety, relative to groups substituted on the double bond carbons. Examples of such groups are ethenyl, propenyl, butenyl, isobutenyl, pentenyl, hexenyl and the like.

As used herein the term “alkoxy” refers to straight or branched chain oxy containing radicals of the formula —OR¹⁰, wherein R¹⁰ is an alkyl group as defined herein. Examples of alkoxy groups encompassed include methoxy, ethoxy, n-propoxy, n-butoxy, isopropoxy, isobutoxy, sec-butoxy, t-butoxy and the like. As used herein the term “alkoxyalkyl” refers to alkyl radicals having one or more alkoxy radicals attached to the alkyl radical, that is, to form monoalkoxyalkyl and dialkoxyalkyl radicals.

As used herein the term “alkoxycarbonylalkyl” refers to a radical of the formula 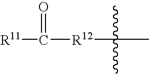
wherein R¹¹ is alkoxy as defined herein and R¹² is alkyl as defined.

As used herein, the term “alkyl” refers to a straight chain or branched chain hydrocarbon radicals having from about 1 to about 10 carbon atoms, and more preferably from about 1 to about 6 carbon atoms. Examples of such alkyl radicals are methyl, ethyl, n-propyl, isopropyl, n-butyl, isobutyl, sec-butyl, t-butyl, pentyl, neopentyl, hexyl, isohexyl, and the like.

As used herein the term “alkylamino” refers to a radical of the formula 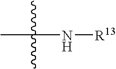
wherein R¹³ is alkyl as defined herein.

As used herein the term “alkylcarbonyl” refers to a radical of the formula 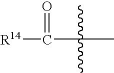
wherein R¹⁴ is alkyl as defined herein.

As used herein term “alkylheteroaryl” refers to a radical of the formula 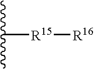
R¹⁵—R¹⁶
wherein R¹⁵ is alkyl as defined herein and R¹⁶ is a heteroaryl as defined herein. As used herein, alkylheteroaryl includes both mono- and poly-alkyl aryl.

As used herein the term “alkylsulfonylalkyl” refers to a radical of the formula 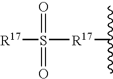
wherein R¹⁷ is alkyl as defined herein.

As used herein the term “alkylthio” refers to a radical of the formula —SR¹⁸ wherein R¹⁸ is alkyl as defined herein.

As used herein the term “alkynyl” refers to acyclic hydrocarbon radicals containing one or more triple bonds and 2 to about 6 carbon atoms, more preferably from 2 to about 3 carbon atoms. Examples of such groups are ethynyl, propynyl, butynyl, pentynyl, hexynyl and the like.

As used herein the term “allyl” refers of a radical of the formula —CH₂CH═CH₂.

As used herein the term “amino” is represented by a radical of the formula —NH₂.

As used herein the term “aminocarbonylalkyl” refers to an alkyl radical as described herein to which is appended an aminocarbonyl (NH₂C(O)—) group.

The term “aryl” means a fully unsaturated mono- or multi-ring carbocycle, including, but not limited to, substituted or unsubstituted phenyl, naphthyl, or anthracenyl.

As used herein the term “benzoyl” refers to the aryl radical C₆H₅—CO—.

As used herein the term “benzyl” refers to the radical

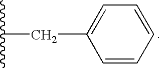

As used herein the term “carboxamide” or “carboxamido” refer to a radical of the formula —CO—NH₂.

As used herein the term “carboxylic acid” refers to the radical —COOH

As used herein the term “carboxyl ester” refers to a radical of the formula —COOR¹⁹ wherein R¹⁹ is selected from the group consisting of hydrogen or alkyl as defined herein.

As used herein, the term “composition” as used herein means a product which results from the mixing or combining of more than one element or ingredient.

As used herein, the term “cyano” is represented by a radical of the formula

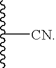

As used herein the term “ethylenedioxy” refers to the radical

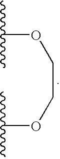

As used herein the term “halogen” or “halo” refers to bromo, chloro, fluoro or iodo.

As used herein the term “haloalkyl” refers to alkyl groups as defined herein substituted with one or more of the same or different halo groups at one or more carbon atom. Examples of haloalkyl groups include trifluoromethyl, dichloroethyl, fluoropropyl and the like.

As used herein the term “haloalkoxy” refers to a radical of the formula —O—R²⁰ wherein R²⁰ is haloalkyl as defined herein.

As used herein the term “heterocyclic” or “heterocycle” means a saturated or unsaturated mono- or multi-ring carbocycle wherein one or more carbon atoms can be replaced by N, S, P, or O. This includes, for example, the following structures: 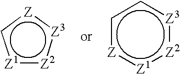
wherein Z, Z¹, Z² or Z³ is C, S, P, O, or N, with the proviso that one of Z, Z¹, Z² or Z³ is other than carbon, but is not O or S when attached to another Z atom by a double bond or when attached to another O or S atom. Furthermore, the optional substituents are understood to be attached to Z, Z¹, Z² or Z³ only when each is C. “Heterocyclic” includes, furanyl, thienyl, pyrrolyl, 2-isopyrrolyl, 3-isopyrrolyl, pyrazolyl, 2-isoimidazolyl, 1,2,3-triazolyl, 1,2,4-triazolyl, 1,2-dithiolyl, 1,3-dithiolyl, 1,2,3-oxathiolyl, isoxazolyl, oxazolyl, thiazolyl, isothiazolyl, 1,2,3-oxadiazolyl, 1,2,4-oxadiazolyl, 1,2,5-oxadiazolyl, 1,3,4-oxadiazolyl, 1,2,3,4-oxatriazolyl, 1,2,3,5-oxatriazolyl, 1,2,3-dioxazolyl, 1,2,4-dioxazolyl, 1,3,2-dioxazolyl, 1,3,4-dioxazolyl, 1,2,5-oxathiazolyl, 1,3-oxathiolyl, 1,2-pyranyl, 1,4-pyranyl, 1,2-pyranonyl, 1,4-pyranonyl, 1,2-dioxinyl, 1,3-dioxinyl, pyridyl, pyridazyl, pyrimidyl, pyrazinyl, piperazyl, 1,3,5-triazinyl, 1,2,4-triazinyl, 1,2,3-triazinyl, 1,2,4-oxazinyl, 1,3,2-oxazinyl, 1,3,6-oxazinyl, 1,2,6-oxazinyl, 1,4-oxazinyl, o-isoxazinyl, p-isoxazinyl, 1,2,5-oxathiazinyl, 1,4-oxazinyl, o-isoxazinyl, p-isoxazinyl, 1,2,5-oxathiainzyl, 1,2,6-oxathiainzyl, 1,4,2-oxadiainzyl, 1,3,5,2-oxadiainzyl, morpholino, azepinyl, oxepinyl, thiepinyl, 1,2,4-diazepinyl, benzofuranyl, isobenzofuranyl, benzothiofuranyl, isobenzothiofuranyl, indolyl, indoleninyl, 2-isobenzazolyl, 1,5-pyrindinyl, pyrano[3,4-b]pyrrolyl, isoindazolyl, indoxazinyl, benzoxazolyl, anthranilyl, 1,2-benzopyranyl, quinolyl, isoquinolyl, cinnolyl, quinazolyl, naphthyridyl, pyrido[3,4-b]pyridyl, pyrido[3,2-b]pyridyl, pyrido[4,3-b]pyridyl, 1,3,2-benzoxazyl, 1,4,2-benzoxazyl, 2,1,3-benzoxazyl, 3,1,4-benzoxazyl, 1,2-benzoisoxazyl, 1,4-benzoisoxazyl, carbazolyl, xanthenyl, acridinyl, purinyl, thiazolidyl, piperidyl, pyrrolidyl, 1,2-dihydroazinyl, 1,4-dihydroazinyl, 1,2,3,6-tetrahydro-1,3-diazinyl, perhydro-1,4-diazinyl, 1,2-thiapyranyl, and 1,4-thiapyranyl.

The terms “hydroxy” and “hydroxyl” as used herein are synonymous and are represented by a radical of the formula

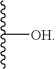

As used herein the term “hydroxyalkyl” refers to a radical of the formula 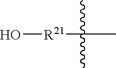
wherein R²¹ is alkyl as defined herein.

As used herein the term “methylenedioxy” refers to the radical

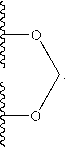

As used herein the term “napthyridinyl” refers to a ring system having the structure

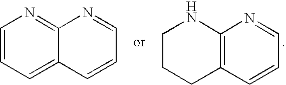

As used herein the term “nitro” is represented by a radical of the formula

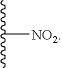

The term “pharmaceutically acceptable carrier”, as used herein means a pharmaceutically acceptable material, composition or vehicle, such as a liquid or solid filler, diluent, excipient, solvent or encapsulating material, involved in carrying or transporting a chemical agent.

As used herein the term “phenethyl” refers to the radical

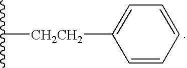

As used herein, the term “selectivity ratio” shall mean the ratio of the inhibition of 50% of the maximum binding (IC₅₀ value) of αᵥβ₃ or αᵥβ₅ over the IC₅₀ value of αᵥβ₆ or α_{IIb}β₃.

As used herein the term “sulfone” refers to a radical of the formula

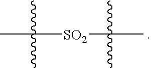

As used herein the term “thioalkyl” refers to a radical of the formula R²³—S— wherein R²³ is alkyl as defined herein.

As used herein, the term “treatment” is meant the medical management of a subject, e.g. an animal or human, with the intent that a prevention, cure, stabilization, or amelioration of the symptoms or condition will result. This term includes active treatment, that is, treatment directed specifically toward improvement of the disorder; palliative treatment, that is, treatment designed for the relief of symptoms rather than the curing of the disorder; preventive treatment, that is, treatment directed to prevention of disorder; and supportive treatment, that is, treatment employed to supplement another specific therapy directed toward the improvement of the disorder. The term “treatment” also includes symptomatic treatment, that is, treatment directed toward constitutional symptoms of the disorder. “Treating” a condition with the compounds of the invention involves administering such a compound, alone or in combination and by any appropriate means, to an animal, cell, lysate or extract derived from a cell, or a molecule derived from a cell.

Abbreviations

The following is a list of abbreviations and the corresponding meanings as used interchangeably herein:
¹H-NMR=proton nuclear magnetic resonance
AcOH=acetic acid
BOC=tert-butoxycarbonyl
BuLi=butyl lithium
Cat.=catalytic amount
CDI=Carbonyldiimidazole
CH₂Cl₂=dichloromethane
CH₃CN=acetonitrile
CH₃I=iodomethane
CHN analysis=carbon/hydrogen/nitrogen elemental analysis
CHNCl analysis=carbon/hydrogen/nitrogen/chlorine elemental analysis
CHNS analysis=carbon/hydrogen/nitrogen/sulfur elemental analysis
DEAD=diethylazodicarboxylate
DIAD=diisopropylazodicarboxylate
DI water=deionized water
DMA=N,N-dimethylacetamide
DMAC=N,N-dimethylacetamide
DMF=N,N-dimethylformamide
EDC=1-(3-dimethylaminopropyl)-3-ethylcarbodiimide hydrochloride
Et=ethyl
Et₂O=diethyl ether
Et₃N=triethylamine
EtOAc=ethyl acetate
EtOH=ethanol
FAB MS=fast atom bombardment mass spectroscopy
g=gram(s)
HOBT=1-hydroxybenzotriazole hydrate
HPLC=high performance liquid chromatography
i-Pr=isopropyl
i-Prop=isopropyl
K₂CO₃=potassium carbonate
KMnO₄=potassium permanganate
KOH=potassium hydroxide
KSCN=potassium thiocyanate
L=Liter
LiOH=lithium hydroxide
Me=methyl
MeOH=methanol
mg=milligram
MgSO₄=magnesium sulfate
ml=milliliter
mL=milliliter
MS=mass spectroscopy
NaH—sodium hydride
NaHCO₃=sodium bicarbonate
NaOH=sodium hydroxide
NaOMe=sodium methoxide
NH₄⁺HCO₂⁻=ammonium formate
NMR=nuclear magnetic resonance
Pd=palladium
Pd/C=palladium on carbon
Ph=phenyl
Pt=platinum
Pt/C=platinum on carbon
RPHPLC=reverse phase high performance liquid chromatography
RT=room temperature
t-BOC=tert-butoxycarbonyl
TFA=trifluoroacetic acid
THF=tetrahydrofuran
TLC—thin layer chromatography
TMS=trimethylsilyl
Δ=heating the reaction mixture
   Indications

In one embodiment, compounds of the present invention are useful for treating an αᵥβ₃ integrin-mediated condition. The integrin identified as αᵥβ₃ (also known as the vitronectin receptor) has been identified as an integrin which plays a role in various conditions or disease states.

It has been reported that the cell surface receptor αᵥβ₃ is the major integrin on osteoclasts responsible for attachment to bone. Osteoclasts cause bone resorption and when such bone resorbing activity exceeds bone forming activity it results in osteoporosis (loss of bone), which leads to an increased number of bone fractures, incapacitation and increased mortality. Antagonists of αᵥβ₃ have been shown to be potent inhibitors of osteoclastic activity in vivo (Fisher et al., Endocrinology, Vol. 132 (1993) 1411-1413). The integrin αᵥβ₃ also is involved in osteopenia (Lark et al., J Bone Miner Res. 2001, 16, 319). Antagonism of αᵥβ₃ leads to decreased bone resorption and therefore restores a normal balance of bone forming and resorbing activity. Thus antagonists of osteoclast αᵥβ₃ are effective inhibitors of bone resorption. Bone resorption conditions include osteopetrosis, osteoporosis, osteomyelitis, hypercalcemia, osteonecrosis, bone loss due to rheumatoid arthritis, periodontal bone loss, immobilization, or prosthetic loosing, Paget's disease, bone cancers (e.g., metastases in bone), osteomalacia, rickets, renal osteodystrophy, and osteopenia brought on by surgery or steroid administration.

In one embodiment, the present invention is useful for treating osteoporosis, Paget's disease, periodontal bone loss and/or osteopenia.

The αᵥβ₃ integrin is also known to mediate angiogenesis, including tumor angiogenesis (Cheresh, Cancer Metastasis Rev., 1991, 10, 3-10 and Brooks, et al., Cell, 1994, 79, 1157). Angiogenesis is characterized by the invasion, migration and proliferation of smooth muscle and endothelial cells. Antagonists of αᵥβ₃ inhibit this process by selectively promoting apoptosis of cells in neovasculature. For example, Brooks et al. (Cell, Vol. 79 (1994) 1157-1164) have demonstrated that antagonists of αᵥβ₃ provide a therapeutic approach for the treatment of neoplasia (inhibition of solid tumor growth) since systemic administration of αᵥβ₃ antagonists causes dramatic regression of various histologically distinct human tumors. Further, mediation of the tumor cell metastatic pathway by interference with the αᵥβ₃ integrin cell adhesion receptor to impede tumor metastasis will be beneficial.

In another embodiment, compounds of the present invention are useful for treating solid tumor growth, tumor angiogenesis, tumor metastasis, and humoral hypercalcemia of malignancy.

The growth of new blood vessels, or angiogenesis, also contributes to pathological conditions such as diabetic retinopathy including macular degeneration (Adamis et al., Amer. J. Ophthal., Vol. 118, (1994) 445-450) and rheumatoid arthritis (Peacock et al., J. Exp. Med., Vol. 175, (1992), 1135-1138). Therefore, αᵥβ₃ antagonists of the present invention will be useful therapeutic agents for treating such conditions associated with neovascularization. In one embodiment, the neovascular conditions include solid tumor angiogenesis, retinopathy including macular degeneration, arthritis, including rheumatoid arthritis, periodontal disease, psoriasis and smooth muscle cell migration (e.g. restenosis and artherosclerosis. In another embodiment, the neovascular conditions are selected from vascular tumors such as haemangioma, neovascularization in the retina, choroid, or cornea, associated with diabetic retinopathy or premature infant retinopathy or macular degeneration and proliferative vitreoretinopathy, rheumatoid arthritis, Crohn's disease, atherosclerosis, ovarian hyperstimulation, psoriasis, endometriosis associated with neovascularization, restenosis subsequent to balloon angioplasty, sear tissue overproduction, for example, that seen in a keloid that forms after surgery, fibrosis after myocardial infarction, or fibrotic lesions associated with pulmonary fibrosis.

The integrin αᵥβ₅ also plays a role in neovascularization. Antagonists of the αᵥβ₅ integrin will inhibit neovascularization and will be useful for treating and preventing angiogenesis metastasis, tumor growth, macular degeneration and diabetic retionopathy. M. C. Friedlander, et al., Science, 270, 1500-1502 (1995) disclose that a monoclonal antibody for αᵥβ₅ inhibits VEFG-induced angogenesis in the rabbit cornea and the chick chorioallantoic membrane model. Therefore, it is useful to antagonize both the αᵥβ₅ and the αᵥβ₃ receptor. Such “mixed αᵥβ₅/αᵥβ₃ antagonists” or “dual αᵥβ₃/αᵥβ₅ antagonists” will be useful for treating or preventing angiogenesis, tumor metastasis, tumor growth, diabetic retinopathy, macular degeneration, atherosclerosis and osteoporosis.

The role of the αᵥβ₃ integrin in smooth muscle cell migration also makes it a therapeutic target for prevention or inhibition of neointimal hyperplasia which is a leading cause of restenosis after vascular procedures (Choi et al., J. Vasc. Surg. Vol. 19(1) (1994) 125-34). In another embodiment, compounds of the present invention prevent or inhibit restenosis.

White (Current Biology, Vol. 3(9)(1993) 596-599) has reported that adenovirus uses αᵥβ₃ for entering host cells. The integrin appears to be required for endocytosis of the virus particle and may be required for penetration of the viral genome into the host cell cytoplasm. Thus compounds of the present invention are therefore useful as antimicrobial agents, and in another embodiment, antiviral agents.

The compounds of the present invention are αᵥβ₃ antagonists and/or dual αᵥβ₃/αᵥβ₅ antagonists and can be used, alone or in combination with other therapeutic agents, in the treatment or modulation of various conditions or disease states described above.

Methods of Treatment

In one embodiment, the present invention relates to a method of selectively inhibiting or antagonizing the αᵥβ₃ integrin and/or the αᵥβ₅ integrin and more specifically relates to a method of inhibiting bone resorption, periodontal disease, osteoporosis, humoral hypercalcemia of malignancy, Paget's disease, tumor metastasis, solid tumor growth (neoplasia), angiogenesis, including tumor angiogenesis, retinopathy including macular degeneration and diabetic retinopathy, arthritis, including rheumatoid arthritis, smooth muscle cell migration and restenosis by administering a therapeutically effective amount of a compound of Formula I to achieve such inhibition together with a pharmaceutically acceptable carrier.

In one embodiment, the present invention is directed towards a method of treating an αᵥβ₃ integrin-mediated condition. In another embodiment, the treatment is ameliorative treatment. In another embodiment, the treatment is palliative treatment. In yet another embodiment, the treatment is preventive treatment.

More specifically it has been found that it is advantageous to administer compounds which are αᵥβ₃ integrin and/or αᵥβ₅ selective and that such selectivity is beneficial in reducing unwanted side-effects.

For the selective inhibition or antagonism of αᵥβ₃ and/or αᵥβ₅ integrins, compounds of the present invention may be administered orally (such as by tablets, capsules [each of which includes sustained release or timed release formulations], pills powders, granules, elixirs, tinctures, suspensions, syrups and emulsions), parenterally, by inhalation spray, topically (e.g., ocular eyedrop), or transdermally (e.g., patch), all in unit dosage formulations containing conventional pharmaceutically acceptable carriers, adjuvants and vehicles. The term parenteral as used herein includes, for example, subcutaneous, intravenous (bolus or infusion), intramuscular, intrasternal, transmuscular infusion techniques or intraperitonally, all using forms well known to those of ordinary skill in the art.

Compounds of the present invention can also be administered via liposomes (e.g., unilamellar vesicles, large unilamellar vesicles, and multilamellar vesicles), and can be formed from a variety of phospholipids. Further, compounds of the present invention can be coupled to an antibody, such as a monoclonal antibody or fragment thereof, or to a soluble polymer for targeted drug delivery.

The compounds of the present invention are administered by any suitable route in the form of a pharmaceutical composition adapted to such a route, and in a dose effective for the treatment intended. Therapeutically effective doses of the compounds required to prevent or arrest the progress of or to treat the medical condition are readily ascertained by one of ordinary skill in the art using preclinical and clinical approaches familiar to the medicinal arts.

Accordingly, the present invention provides a method of treating conditions mediated by selectively inhibiting or antagonizing the αᵥβ₃ and/or αᵥβ₅ cell surface receptor which method comprises administering a therapeutically effective amount of a compound selected from the class of compounds depicted in the above formulas, wherein one or more compound is administered in association with one or more non-toxic, pharmaceutically acceptable carriers and/or diluents and/or adjuvants (collectively referred to herein as “carrier” materials) and if desired other active ingredients. More specifically, the present invention provides a method for selective antagonism of the αᵥβ₃ and/or αᵥβ₅ cell surface receptors over α_{IIb}β₃ or αᵥβ₆ integrin receptors. Most preferably the present invention provides a method for inhibiting bone resorption, treating osteoporosis, inhibiting humoral hypercalcemia of malignancy, treating Paget's disease, inhibiting tumor metastasis, inhibiting neoplasia (solid tumor growth), inhibiting angiogenesis including tumor angiogenesis, treating retinopathy including macular degeneration and diabetic retinopathy, inhibiting arthritis, psoriasis and periodontal disease, and inhibiting smooth muscle cell migration including restenosis.

Based upon standard laboratory experimental techniques and procedures well known and appreciated by those skilled in the art, as well as comparisons with compounds of known usefulness, the compounds of Formulas I-IX can be used in the treatment of patients suffering from the above pathological conditions. One skilled in the art will recognize that selection of the most appropriate compound of the invention is within the ability of one with ordinary skill in the art and will depend on a variety of factors including assessment of results obtained in standard assay and animal models.

Treatment of a patient afflicted with one of the pathological conditions comprises administering to such a patient an amount of compound of Formulas I-VII which is therapeutically effective in controlling the condition or in prolonging the survivability of the patient beyond that expected in the absence of such treatment. As used herein, the term “inhibition” of the condition refers to slowing, interrupting, arresting or stopping the condition and does not necessarily indicate a total elimination of the condition. It is believed that prolonging the survivability of a patient, beyond being a significant advantageous effect in and of itself, also indicates that the condition is beneficially controlled to some extent.

As stated previously, the compounds of the invention can be used in a variety of biological, prophylactic or therapeutic areas. It is contemplated that these compounds are useful in prevention or treatment of any disease state or condition wherein the αᵥβ₃ and/or αᵥβ₅ integrin plays a role.

The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment of the above-indicated conditions.

Oral delivery of an αᵥβ₃ and/or αᵥβ₅ inhibitor of the present invention can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

Oral dosages of the present invention, when used for the indicated effects, will range between about 0.01 mg per kg of body weight per day (mg/kg/day) to about 100 mg/kg/day, and in another embodiment 0.01 to 10 mg/kg/day, and in yet another embodiment 0.1 to 1.0 mg/kg/day. For oral administration, the compositions are in one embodiment provided in the form of tablets containing 0.01, 0.05, 0.1, 0.5, 1.0, 2.5, 5.0, 10.0, 15.0, 25.0, 50.0, 100, 200 and 500 milligrams of the active ingredient for the symptomatic adjustment of the dosage to the patient to be treated. A medicament typically contains from about 0.01 mg to about 500 mg of the active ingredient, in one embodiment, from about 1 mg to about 100 mg of active ingredient. Intravenously, the most typical doses will range from about 0.1 to about 10 mg/kg/minute during a constant rate infusion.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will be continuous rather than intermittent throughout the dosage regiment.

For administration to a mammal in need of such treatment, the compounds in a therapeutically effective amount are ordinarily combined with one or more adjuvants appropriate to the indicated route of administration. The compounds may be admixed with, for example, lactose, sucrose, starch powder, cellulose esters of alkanoic acids, cellulose alkyl esters, talc, stearic acid, magnesium stearate, magnesium oxide, sodium and calcium salts of phosphoric and sulphuric acids, gelatin, acacia, sodium alginate, polyvinylpyrrolidone, and/or polyvinyl alcohol, and tableted or encapsulated for convenient administration. Alternatively, the compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

The pharmaceutical compositions useful in the present invention may be subjected to conventional pharmaceutical operations such as sterilization and/or may contain conventional pharmaceutical adjuvants such as preservatives, stabilizers, wetting agents, emulsifiers, buffers, etc.

Pharmaceutical compositions suitable for oral administration can be presented in discrete units, such as capsules, cachets, lozenges, or tablets, each containing a predetermined amount of at least one compound of the present invention; as a powder or granules; as a solution or a suspension in an aqueous or non-aqueous liquid; or as an oil-in-water or water-in-oil emulsion. As indicated, such compositions can be prepared by any suitable method of pharmacy which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product. For example, a tablet can be prepared by compressing or molding a powder or granules of the compound, optionally with one or more assessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

Pharmaceutical compositions suitable for buccal (sub-lingual) administration include lozenges comprising a compound of the present invention in a flavored base, usually sucrose, and acacia or tragacanth, and pastilles comprising the compound in an inert base such as gelatin and glycerin or sucrose and acacia.

Pharmaceutical compositions suitable for parenteral administration conveniently comprise sterile aqueous preparations of a compound of the present invention. These preparations are preferably administered intravenously, although administration can also be effected by means of subcutaneous, intramuscular, or intradermal injection. Such preparations can conveniently be prepared by admixing the compound with water and rendering the resulting solution sterile and isotonic with the blood. Injectable compositions according to the invention will generally contain from 0.1 to 5% w/w of a compound disclosed herein.

Pharmaceutical compositions suitable for topical application to the skin preferably take the form of an ointment, cream, lotion, paste, gel, spray, aerosol, or oil. Carriers which can be used include Vaseline, lanolin, polyethylene glycols, alcohols, and combinations of two or more thereof. The active compound is generally present at a concentration of from 0.1 to 15% w/w of the composition, for example, from 0.5 to 2%.

Transdermal administration is also possible. Pharmaceutical compositions suitable for transdermal administration can be presented as discrete patches adapted to remain in intimate contact with the epidermis of the recipient for a prolonged period of time. Such patches suitably contain a compound of the present invention in an optionally buffered, aqueous solution, dissolved and/or dispersed in an adhesive, or dispersed in a polymer. A suitable concentration of the active compound is about 1% to 35%, preferably about 3% to 15%. As one particular possibility, the compound can be delivered from the patch by electrotransport or iontophoresis, for example, as described in Pharmaceutical Research, 3(6), 318 (1986).

In any case, the amount of active ingredient that can be combined with carrier materials to produce a single dosage form to be administered will vary depending upon the host treated and the particular mode of administration.

The solid dosage forms for oral administration including capsules, tablets, pills, powders, and granules noted above comprise one or more compounds of the present invention admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

The term “therapeutically effective amount” shall mean that amount of drug or pharmaceutical agent that will elicit the biological or medical response of a tissue, system or animal that is being sought by a researcher or clinician.

### EXAMPLES

The general synthetic sequences for preparing the compounds useful in the present invention are outlined in Examples 1-4. Both an explanation of, and the actual procedures for, the various embodiments of the present invention are described where appropriate. The following Examples are intended to be merely illustrative of the present invention, and not limiting thereof in either scope or spirit. Those with skill in the art will readily understand that known variations of the conditions and processes described in the Examples can be used to synthesize the compounds of the present invention.

### Example 1

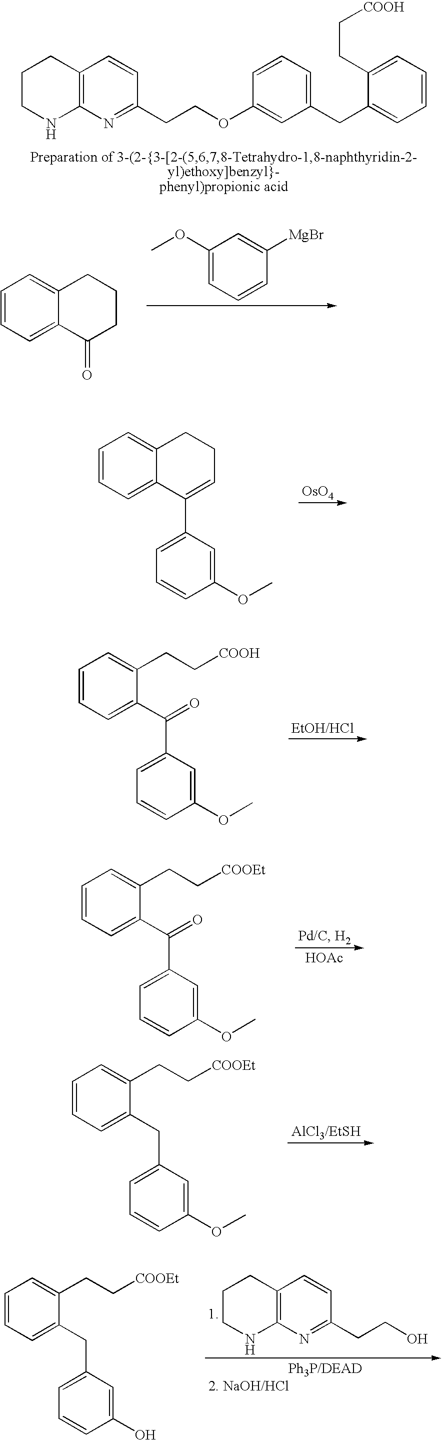

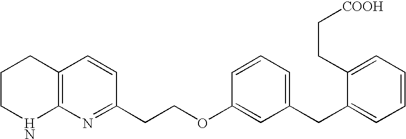

### Example 2

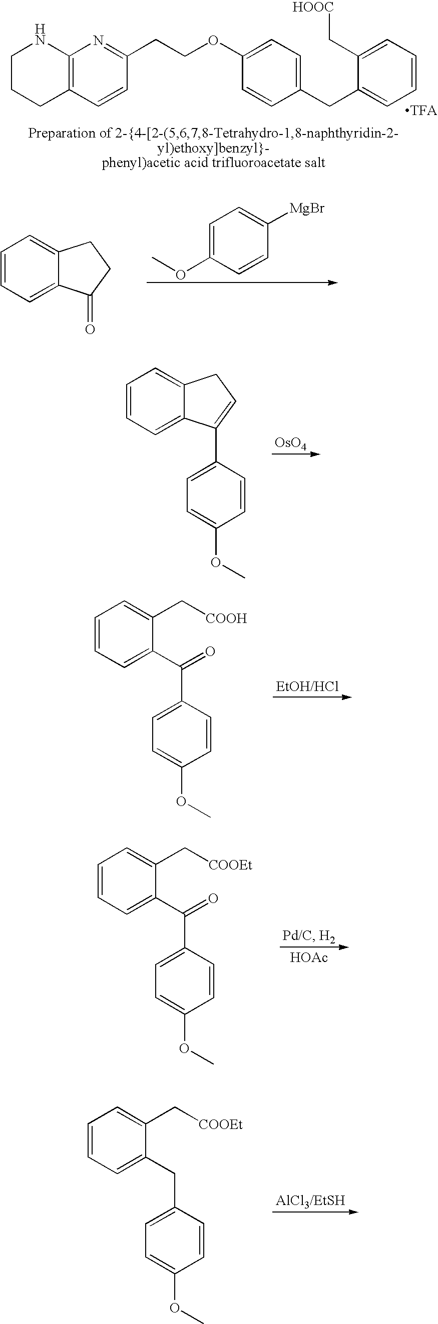

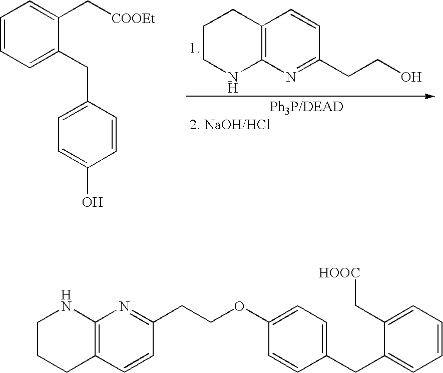

Ethyl 2-[2-(4-hydroxybenzyl)phenyl]acetate was prepared as described in Example 1 steps 1-5, starting from 1-indanone and p-methoxyphenyl magnesium bromide. A solution of DEAD (1.011 g, 5.81 mmol) in DMF (10 mL) was added to a solution of ethyl 2-[2-(4-hydroxybenzyl)phenyl]acetate (0.78 g, 2.89 mmol) and triphenylphosphine (1.51 g, 5.76 mmol) and the reaction mixture was stirred at rt for 18 h. The reaction mixture was concentrated and was purified on HPLC (38% CH₃CN in water containing 0.01% TFA) to afford the ester of the desired product. The ester was hydrolyzed (ethanol/water and sodium hydroxide) and was purified on HPLC to afford 0.47 g (31%) of the TFA salt of the desired product as oil. ¹H NMR (CDCL3) δ 7.58 (d, 1H, J=7.2 Hz), 6.99-7.21 (m, 6H), 6.80 (d, 2H, J=8.7 Hz), 6.70 (d, 1H, J=7.4 Hz), 4.23 (t, 2H, J=5.5 Hz), 3.94 (s, 2H), 3.54 (t, 2H, J=5.5 Hz), 3.11 (t, 2H, J=5.9 Hz), 2.78-2.86 (m, 2H), 2.32-2.38 (m, 2H), 1.89-1.94 (m, 2H). Anal. Calcd for C₂₅H₂₅N₂O₃: Mol. Wt. 403.2016 (M+H). Found: 403.2020 (M+H), HRMS).

### Example 3

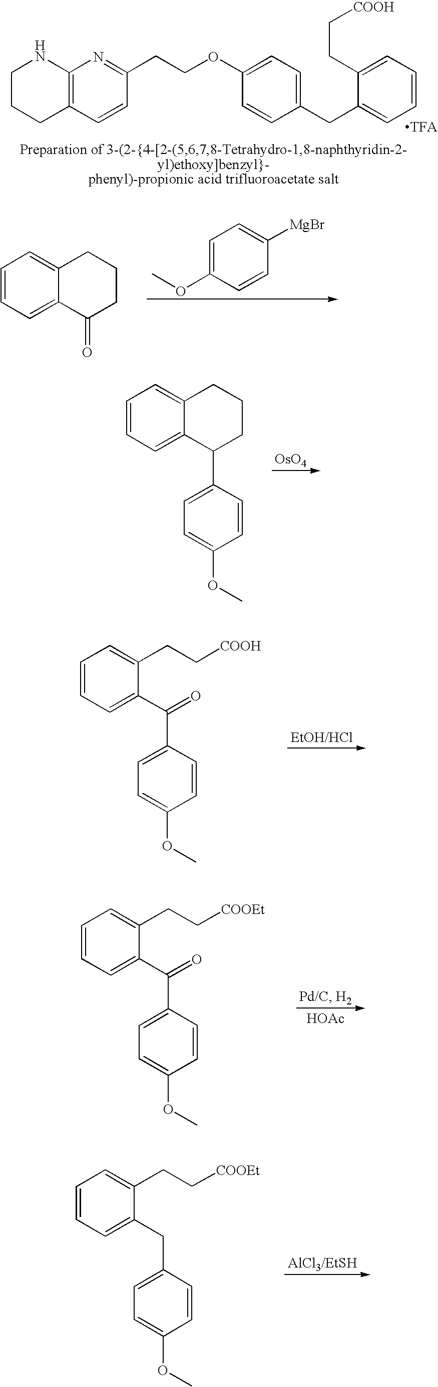

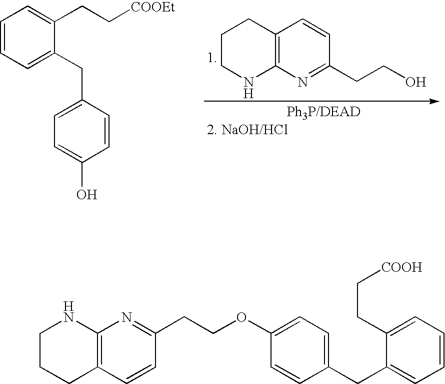

Ethyl 3-[2-(4-hydroxybenzyl)phenyl]propionate was prepared as described in Example 1, steps 1-5, starting from 1-decalone and p-methoxyphenyl magnesium bromide. A solution of DEAD (1.01 g, 5.81 mmol) and 5,6,7,8-tetrahydro-1,8-naphthyridine-2-ethanol (1.03 g, 5.79 mmol) in DMF (10 mL) was added to a solution of ethyl 2-[2-(4-hydroxybenzyl)phenyl]acetate (0.82 g, 2.89 mmol) and triphenylphosphine (1.51 g, 5.76 mmol) and the reaction mixture was stirred at rt for 18 h. The reaction mixture was concentrated and then was purified on HPLC (38% CH₃CN in water containing 0.01% TFA) to afford the ester of the desired product. The ester was hydrolyzed (ethanol/water and sodium hydroxide) and was purified on HPLC to afford 0.47 g (31%) of the TFA salt of the desired product as oil. ¹H NMR (CDCL₃) δ 7.58 (m, 1H), 6.98-7.15 (m, 6H), 6.79 (d, 2H, J=8.6 Hz), 6.70 (d, 1H, J=7.4 Hz), 4.23 (t, 2H, J=6.1 HZ), 3.94-3.96 (m, 2H), 3.59 (s, 1H), 3.47 (t, 2H, J=5.5 Hz), 3.33 (S, 1H), 3.11 (t, 2H, J=5.9 Hz), 2.78-2.86 (m, 4H), 2.31-2.39 (m, 2H), 1.89-1.94 (m, 2H). Anal. Calcd for C₂₆H₂₈N₂O₃: Mol. Wt. 417.2173 (M+H). Found: 417.2162 (M+H, HRMS).

### Example 4

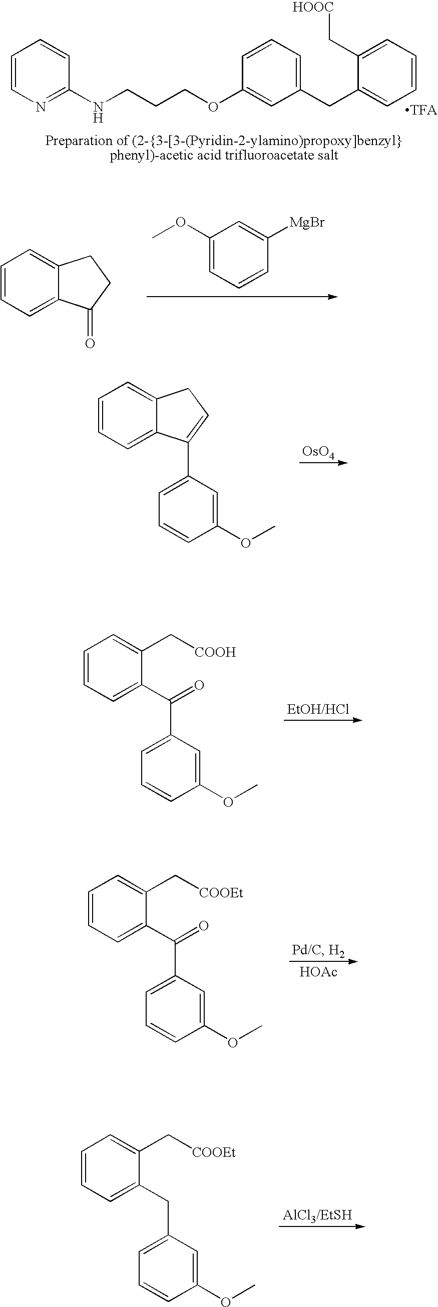

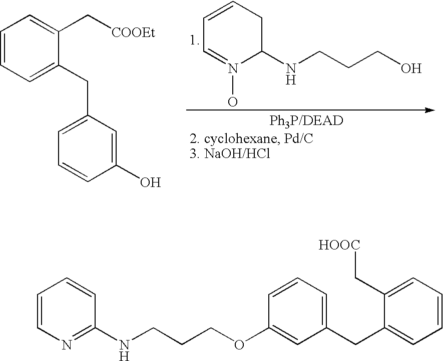

Ethyl 2-[2-(3-hydroxybenzyl)phenyl]acetate was prepared as described in Example 1, steps 1-5, starting from I-indanone and m-methoxyphenyl magnesium bromide. A solution of DEAD (5.8 g, 33.34 mmol) and 2-[(3-hydroxy-1-propyl)-amino]pyridine-N-oxide (5.6 g, 33.34 mmol) in DMF (50 mL) was added to a solution of ethyl 2-[2-(3-hydroxybenzyl)phenyl]acetate (4.50 g, 16.67 mmol) and triphenylphosphine (9.20 g, 35 mmol) in DMF (100 mL) and the reaction mixture was stirred at rt for 18 h. The reaction mixture was concentrated and the residue was purified by HPLC to afford 6 g of the desired product. A mixture of the product (6.0 g), Palladium/Carbon (4 g, 10%) and cyclohexene (9 mL) in ethanol (150 mL) was heated at reflux for 18 h. The catalyst was removed by filtration and the filtrate was concentrated to afford a residue, which was carried to the next stage without further purification. The residue in ethanol (100 mL) was added aq. Sodium hydroxide till basic (2.5 N). After 24 h, the reaction mixture was concentrated, acidified with TFA and was purified by HPLC to afford the desired product. ¹H NMR (CD₃OD) δ 7.75-7.86 (m, 2H), 7.04-7.23 (m, 6H), 6.6-6.84 (m, 4H), 3.97-4.07 (m, 4H), 3.55-3.60 (m, 4H), 2.02-2.17 (m, 2H). Anal. Calcd for C₂₃H₂₄N₂O₃: Mol. Wt, 377.1865 (M+H). Found: Mol. Wt, 377.1866 (M+H, HRMS).

The activity of the compounds of the present invention was tested in the following assays. In one embodiment, compounds of the present invention antagonize the αᵥβ₃ integrin with an IC₅₀ of 0.1 nM to 100 μM in the 293-cell assay. In another embodiment, compounds of the present invention antagonize the αᵥβ₃ integrin with an IC₅₀ of 0.1 nM to 0.2 μM in the 293-cell assay. Similarly these compounds also antagonized the αᵥβ₅ integrin with an IC₅₀ of about 0.1 nM to about 100 μM in the cell adhesion assay, and in another embodiment, from 0.1 nM to 0.2 μM.

In yet another embodiment, the compounds of the present invention also antagonized the IIb-IIIa integrin with an IC₅₀ of greater than about 1 μmol/Lμ μM. In a further embodiment, compounds of the present invention antagonized the αᵥβ₆ integrin with an IC₅₀ of greater than about 1 μM in the HT-29 cell-based adhesion assay. In another embodiment, the compounds further have a selectivity ratio of αᵥβ₃ integrin antagonism over the IIb3a integrin antagonism of at least about 10, and in another embodiment, of at least 100. In another embodiment, the compounds further have a selectivity ratio of αᵥβ₃ integrin antagonism over the αᵥβ₆ integrin antagonism of at least about 10, and in another embodiment, of at least 100.

### Vitronectin Adhesion Assay

### Materials

Human vitronectin receptors αᵥβ₃ and αᵥβ₅ were purified from human placenta as previously described (Pytela et al., Methods in Enzymology, 144:475-489 (1987)). Human vitronectin was purified from fresh frozen plasma as previously described (Yatohgo et al., Cell Structure and Function, 13:281-292 (1988)). Biotinylated human vitronectin was prepared by coupling NHS-biotin from Pierce Chemical Company (Rockford, Ill.) to purified vitronectin as previously described (Charo et al., J. Biol. Chem., 266(3):1415-1421 (1991)). Assay buffer, OPD substrate tablets, and RIA grade BSA were obtained from Sigma (St. Louis, Mo.). Anti-biotin antibody was obtained from Sigma (St. Louis, Mo.). Nalge Nunc-Immuno microtiter plates were obtained from Nalge Company (Rochester, N.Y.).

### Methods

### Solid Phase Receptor Assays

This assay was essentially the same as previously reported (Nilya et al., Blood, 70:475-483 (1987)). The purified human vitronectin receptors αᵥβ₃ and αᵥβ₅ were diluted from stock solutions to 1.0 μg/mL in Tris-buffered saline containing 1.0 mM Ca⁺⁺, Mg⁺⁺, and Mn⁺⁺, pH 7.4 (TBS⁺⁺⁺). The diluted receptors were immediately transferred to Nalge Nunc-Immuno microtiter plates at 100 μL/well (100 ng receptor/well). The plates were sealed and incubated overnight at 4° C. to allow the receptors to bind to the wells. All remaining steps were at room temperature. The assay plates were emptied and 200 μL of 1% RIA grade BSA in TBS⁺⁺⁺ (TBS⁺⁺⁺/BSA) were added to block exposed plastic surfaces. Following a 2 hour incubation, the assay plates were washed with TBS⁺⁺⁺ using a 96 well plate washer. Logarithmic serial dilution of the test compound and controls were made starting at a stock concentration of 2 mM and using 2 nM biotinylated vitronectin in TBS⁺⁺⁺/BSA as the diluent. This premixing of labeled ligand with test (or control) ligand, and subsequent transfer of 50 μL aliquots to the assay plate was carried out with a CETUS Propette robot; the final concentration of the labeled ligand was 1 nM and the highest concentration of test compound was 1.0×10⁻⁴ M. The competition occurred for two hours after which all wells were washed with a plate washer as before. Affinity purified horseradish peroxidase labeled goat anti-biotin antibody was diluted 1:2000 in TBS⁺⁺⁺/BSA and 125 μL was added to each well. After 45 minutes, the plates were washed and incubated with OPD/H₂O₂ substrate in 100 mM/L Citrate buffer, pH 5.0. The plate was read with a microtiter plate reader at a wavelength of 450 nm and when the maximum-binding control wells reached an absorbance of about 1.0, the final A₄₅₀ were recorded for analysis. The data were analyzed using a macro written for use with the EXCEL spreadsheet program. The mean, standard deviation, and % CV were determined for duplicate concentrations. The mean A₄₅₀ values were normalized to the mean of four maximum-binding controls (no competitor added)(B-MAX). The normalized values were subjected to a four parameter curve fit algorithm (Rodbard et al., Int. Atomic Energy Agency, Vienna, pp 469 (1977)), plotted on a semi-log scale, and the computed concentration corresponding to inhibition of 50% of the maximum binding of biotinylated vitronectin (IC₅₀) and corresponding R² was reported for those compounds exhibiting greater than 50% inhibition at the highest concentration tested; otherwise the IC₅₀ is reported as being greater than the highest concentration tested. β-[[2-[[5-[(aminoiminomethyl)amino]-1-oxopentyl]amino]-1-oxoethyl]amino]-3-pyridinepropanoic acid (U.S. Pat. No. 5,602,155 Example 1) which is a potent αᵥβ₃ antagonist (IC₅₀ in the range 3-10 nM) was included on each plate as a positive control.

### Purified IIb/IIIa Receptor Assay

### Materials

Human fibrinogen receptor (IIb/IIIa) was purified from outdated platelets. (Pytela, R., Pierschbacher, M. D., Argraves, S., Suzuki, S., and Rouslahti, E. “Arginine-Glycine-Aspartic acid adhesion receptors”, Methods in Enzymology 144 (1987):475-489). Human vitronectin was purified from fresh frozen plasma as described in Yatohgo, T., Izumi, M., Kashiwagi, H., and Hayashi, M., “Novel purification of vitronectin from human plasma by heparin affinity chromatography,” Cell Structure and Function 13 (1988):281-292. Biotinylated human vitronectin was prepared by coupling NHS-biotin from Pierce Chemical Company (Rockford, Ill.) to purified vitronectin as previously described. (Charo, I. F., Nannizzi, L., Phillips, D. R., Hsu, M. A., Scarborough, R. M., “Inhibition of fibrinogen binding to GP IIb/IIIa by a GP IIIa peptide”, J. Biol. Chem. 266(3)(1991): 1415-1421.) Assay buffer, OPD substrate tablets, and RIA grade BSA were obtained from Sigma (St. Louis, Mo.). Anti-biotin antibody was obtained from Sigma (St. Louis, Mo.). Nalge Nunc-Immuno microtiter plates were obtained from (Rochester, N.Y.). ADP reagent was obtained from Sigma (St. Louis, Mo.).

### Solid Phase Receptor Assays

This assay is essentially the same reported in Nilya, K., Hodson, E., Bader, R., Byers-Ward, V. Koziol, J. A., Plow, E. F. and Ruggeri, Z. M., “Increased surface expression of the membrane glycoprotein IIb/IIIa complex induced by platelet activation: Relationships to the binding of fibrinogen and platelet aggregation”, Blood 70 (1987):475-483. The purified human fibrinogen receptor (IIb/IIIa) was diluted from stock solutions to 1.0 μg/mL in Tris-buffered saline containing 1.0 mM Ca⁺⁺, Mg⁺⁺, and Mn⁺⁺, pH 7.4 (TBS⁺⁺⁺). The diluted receptor was immediately transferred to Nalge Nunc-Immuno microtiter plates at 100 μL/well (100 ng receptor/well). The plates were sealed and incubated overnight at 4° C. to allow the receptors to bind to the wells. All remaining steps were at room temperature. The assay plates were emptied and 200 μL of 1% RIA grade BSA in TBS⁺⁺⁺ (TBS⁺⁺⁺/BSA) were added to block exposed plastic surfaces. Following a 2 hour incubation, the assay plates were washed with TBS⁺⁺⁺ using a 96 well plate washer. Logarithmic serial dilution of the test compound and controls were made starting at a stock concentration of 2 mM and using 2 nM biotinylated vitronectin in TBS⁺⁺⁺/BSA as the diluent. This premixing of labeled ligand with test (or control) ligand, and subsequent transfer of 50 μL aliquots to the assay plate was carried out with a CETUS Propette robot; the final concentration of the labeled ligand was 1 nM and the highest concentration of test compound was 1.0×10⁻⁴ M. The competition occurred for two hours after which all wells were washed with a plate washer as before. Affinity purified horseradish peroxidase labeled goat anti-biotin antibody was diluted 1:2000 in TBS⁺⁺⁺/BSA and 125 μL were added to each well. After 45 minutes, the plates were washed and incubated with ODD/H₂O₂ substrate in 100 mM/L citrate buffer, pH 5.0. The plate was read with a microtiter plate reader at a wavelength of 450 nm and when the maximum-binding control wells reached an absorbance of about 1.0, the final A₄₅₀ were recorded for analysis. The data were analyzed using a macro written for use with the EXCELJ spreadsheet program. The mean, standard deviation, and % CV were determined for duplicate concentrations. The mean A₄₅₀ values were normalized to the mean of four maximum-binding controls (no competitor added)(B-MAX). The normalized values were subjected to a four parameter curve fit algorithm, (Robard et al., Int. Atomic Energy Agency, Vienna, pp 469 (1977)), plotted on a semi-log scale, and the computed concentration corresponding to inhibition of 50% of the maximum binding of biotinylated vitronectin (IC₅₀) and corresponding R² was reported for those compounds exhibiting greater than 50% inhibition at the highest concentration tested; otherwise the IC₅₀ is reported as being greater than the highest concentration tested. ?-[[2-[[5-[(aminoimino-methyl)amino]-1-oxopentyl]amino]-1-oxoethyl]amino]-3-pyridine propanoic acid, bistrifluoroacetate salt (U.S. Pat. No. 5,602,155 Example 1) which is a potent IIb/IIIa antagonist (IC₅₀ in the range 8-18 nM) was included on each plate as a positive control.

### Human Platelet Rich Plasma Assays

Healthy aspirin free donors were selected from a pool of volunteers. The harvesting of platelet rich plasma and subsequent ADP induced platelet aggregation assays were performed as described in Zucker, M. B., “Platelet Aggregation Measured by the Photometric Method”, Methods in Enzymology 169 (1989):117-133. Standard venipuncture techniques using a butterfly allowed the withdrawal of 45 mL of whole blood into a 60 mL syringe containing 5 mL of 3.8% trisodium citrate. Following thorough mixing in the syringe, the anti-coagulated whole blood was transferred to a 50 mL conical polyethylene tube. The blood was centrifuged at room temperature for 12 minutes at 200×g to sediment non-platelet cells. Platelet rich plasma was removed to a polyethylene tube and stored at room temperature until used. Platelet poor plasma was obtained from a second centrifugation of the remaining blood at 2000×g for 15 minutes. Platelet counts are typically 300,000 to 500,000 per microliter. Platelet rich plasma (0.45 mL) was aliquoted into siliconized cuvettes and stirred (1100 rpm) at 37° C. for 1 minute prior to adding 50 uL of pre-diluted test compound. After 1 minute of mixing, aggregation was initiated by the addition of 50 uL of 200 uM ADP. Aggregation was recorded for 3 minutes in a Payton dual channel aggregometer (Payton Scientific, Buffalo, N.Y.). The percent inhibition of maximal response (saline control) for a series of test compound dilutions was used to determine a dose response curve. All compounds were tested in duplicate and the concentration of half-maximal inhibition (IC₅₀) was calculated graphically from the dose response curve for those compounds which exhibited 50% or greater inhibition at the highest concentration tested; otherwise, the IC₅₀ is reported as being greater than the highest concentration tested.

### Cell Assays for Potency and Selectivity

While the β₃ subunit of αᵥβ₃ is only known to complex with αᵥ or α_{IIb}, the ax subunit complexes with multiple β subunits. The three αᵥ integrins most homologous with αᵥβ₃ are αᵥβ₁, αᵥβ₅ and αᵥβ₆, with 43%, 56% and 47% amino acid identity in the P subunits, respectively. To evaluate the selectivity of compounds between the integrins αᵥβ₃ and αᵥβ₆, cell-based assays were established using the 293 human embryonic kidney cell line. 293 cells express αᵥβ₁, but little to no detectable αᵥβ₃ or αᵥβ₆. cDNAs for β₃ and β₆ were transfected separately into 293 cells to generate 293-β3 and 293-β6 cells, respectively. High surface expression of αᵥβ₃ and αᵥβ₆ was confirmed by flow cytometry. Conditions were established for each cell line in which cell adhesion to immobilized human vitronectin was mediated by the appropriate integrin, as determined by a panel of integrin-specific, neutralizing monoclonal antibodies. Briefly, cells were incubated with inhibitor in the presence of 200 uM Mn²⁺, allowed to adhere to immobilized vitronectin, washed, and adherent cells are detected endogenous alkaline phosphatase and para-nitrophenyl phosphate. An 8-point dose-response curve using either 10-fold or 3-fold dilutions of compound was evaluated by fitting a four-parameter logistic, nonlinear model (using SAS).

To evaluate compound potency for membrane-bound αᵥβ₆ an additional cell-based adhesion assay was established using the HT-29 human colon carcinoma cell line. High surface expression of αᵥβ₆ on HT-29 cells was confirmed by flow cytometry. Conditions were established in which cell adhesion to immobilized human latency associated peptide (LAP) was mediated by the αᵥβ₆, as determined by a panel of integrin-specific, neutralizing monoclonal antibodies. Briefly, cells were incubated with inhibitor in the presence of 200 uM Mn²⁺, allowed to adhere to immobilized LAP, washed, and adherent cells are detected by quantifying endogenous alkaline phosphatase using para-nitrophenyl phosphate. An 8-point dose-response curve using either 10-fold or 3-fold dilutions of compound was evaluated by fitting a four-parameter logistic, nonlinear model (using SAS). The compounds evaluated were relatively ineffective at inhibition of αᵥβ₆-mediated cell adhesion. The selective antagonism of the αᵥβ₃ integrin is viewed as desirable in this class of compounds, as αᵥβ₆ may also play a role in normal physiological processes of tissue repair and cellular turnover that routinely occur in the skin and pulmonary tissue.

What is claimed is:

1. A compound having the structure of Formula I: 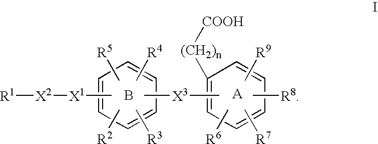
or a pharmaceutically acceptable salt, ester or tautomer thereof; wherein
A and B are phenyl;
n is an integer from 1 to 3;
X¹ is selected from O, NR, S, SO, SO₂, CHR and CH₂, wherein R is selected from the group consisting of hydrogen, aryl, and heterocyclyl;
X² is selected from the group consisting of alkyl, alkylamino, aminoalkyl, alkylaminoalkyl, alklthio, thioalkyl, alkylthioalkyl, alkylsulfonyl, sulfonylalkyl, alkylsulfonylalkyl, oxyalkyl, alkoxyalkyl, and alkoxy;
X³ is C₁-C₆ alkyl or aryl substituted with one or more substituents selected from the group consisting of hydrogen, alkyl, alkenyl, alkoxy, oxyalkyl, alkoxyalkyl, alkoxycarbonylalkyl, alkylamino, alkylcarbonyl, alkylheteroaryl, alkylsulfonylalkyl, alkylthio, alkynyl, aminocarbonylalkyl, cyano, dialkylamino, halo, haloalkoxy, haloalkyl, hydroxy and hydroxyalkyl;
R¹ is selected from the group consisting of pyridinyl and napthyridinyl, wherein either is optionally substituted with a substituent selected from the group consisting of hydrogen, alkyl, halo, and amino;
R², R³, R⁴, R⁵, R⁶, R⁷, R⁸ and R⁹ are independently selected from the group consisting of hydrogen, alkyl, alkenyl, alkoxy, alkoxyalkyl, alkoxycarbonylalkyl, alkylamino, alkylcarbonyl, alkylheteroaryl, alkylsulfonylalkyl, alkylthio, alkynyl, aminocarbonylalkyl, cyano, dialkylamino, halo, haloalkoxy, haloalkyl, hydroxy and hydroxyalkyl; and
wherein X³ is independently meta- or para- to the X¹ of the B ring, and wherein X³ is further ortho-, meta-, or para- to the carboxylic acid chain of the A ring.

2. The compound of claim 1 wherein:
n is an integer from 1 to 2;
X¹ is selected from O, NR, or CH₂, wherein R is hydrogen;
X² is C₁-C₆ alkyl or C₁-C₆ alkylamino;
X³ is —CH₂—; and
R², R³, R⁴, R⁵, R⁶, R⁷, R⁸ and R⁹ are independently selected from hydrogen, C₁-C₆ alkyl, alkenyl, alkynyl, alkoxy, halo, haloalkyl and hydroxy.

3. The compound of claim 1 wherein:
n is 1 or 2;
X¹ is O;
X² is C₁-C₆ alkyl or C₁-C₆ alkylamino;
X³ is —CH₂—; and
R², R³R⁴, R⁵, R⁶, R⁷, R⁸ and R⁹ are independently hydrogen.

4. The compound of claim 3 wherein X² is ethyl or propyl.

5. A compound having the structure of formula V: 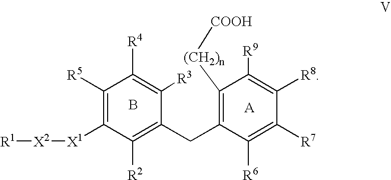
or a pharmaceutically acceptable salt, ester or tautomer thereof, wherein:
n is an integer from 1 to 3;
X¹ is selected from O, NR, S, SO, SO₂, CHR and CH₂, wherein R is selected from the group consisting of hydrogen, aryl, and heterocyclyl;
X² is selected from the group consisting of alkyl, alkylamino, aminoalkyl, alkylaminoalkyl, alklthio, thioalkyl, alkylthioalkyl, alkylsulfonyl, sulfonylalkyl, alkylsulfonylalkyl, oxyalkyl, alkoxyalkyl, and alkoxy;
R¹ is selected from the group consisting of pyridinyl and napthyridinyl;
R², R³R⁴, R⁵, R⁶, R⁷, R⁸ and R⁹ are independently selected from the group consisting of hydrogen, alkyl, alkenyl, alkoxy, alkoxyalkyl, alkoxycarbonylalkyl, alkylamino, alkylcarbonyl, alkylheteroaryl, alkylsulfonylalkyl, alkylthio, alkynyl, aminocarbonylalkyl, cyano, dialkylamino, halo, haloalkoxy, haloalkyl, hydroxy and hydroxyalkyl; and
R¹ is attached meta- or para- to the methylene bridge.

6. The compound of claim 5 wherein:
n is an integer from 1 to 2;
X¹ is selected from the group consisting of O, NR and CH₂ wherein R is hydrogen;
X² is C₁-C₆ alkyl or C₁-C₆ alkylamino;
R², R³R⁴, R⁵, R⁶, R⁷, R⁸ and R⁹ are independently selected from hydrogen, C₁-C₆ alkyl, alkenyl, alkynyl, alkoxy, halo, haloalkyl and hydroxy.

7. The compound of claim 5, wherein:
n is 1 or 2;
X¹ is O;
X² is C₁-C₆ alkyl or C₁-C₆ alkylamino; and
R², R³R⁴, R⁵, R⁶, R⁷, R⁸ and R⁹ are independently hydrogen.

8. A compound having the structure of formula VI: 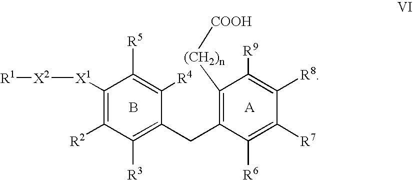
or a pharmaceutically acceptable salt, ester or tautomer thereof, wherein:
n is an integer from 1 to 3;
X¹ is selected from O, NR, S, SO, SO₂, CHR and CH₂, wherein;
R is selected from the group consisting of hydrogen, aryl, and heterocyclyl;
X² is selected from the group consisting of alkyl, alkylamino, aminoalkyl, alkylaminoalkyl, alklthio, thioalkyl, alkylthioalkyl, alkylsulfonyl, sulfonylalkyl, alkylsulfonylalkyl, oxyalkyl, alkoxyalkyl, and alkoxy;
R¹ is selected from the group consisting of pyridinyl and napthyridinyl;
R², R³, R⁴, R⁵, R⁶, R⁷, R⁸ and R⁹ are independently selected from hydrogen, alkyl, alkenyl, alkoxy, alkoxyalkyl, alkoxycarbonylalkyl, alkylamino, alkylcarbonyl, alkylheteroaryl, alkylsulfonylalkyl, alkylthio, alkynyl, aminocarbonylalkyl, cyano, dialkylamino, halo, haloalkoxy, haloalkyl, hydroxy and hydroxyalkyl; and
X¹ of ring B is attached meta- or para- to X³.

9. The compound of claim 8, wherein:
n is an integer from 1 to 2;
X¹ is selected from the group consisting of O, NR and CH₂ wherein R is hydrogen;
X² is C₁-C₆ alkyl or C₁-C₆ alkylamino; and
R², R³, R⁴, R⁵, R⁶, R⁷, R⁸ and R⁹ are independently selected from hydrogen, C₁-C₆ alkyl, alkenyl, alkynyl, alkoxy, halo, haloalkyl and hydroxy.

10. The compound of claim 8, wherein:
n is an integer from 1 to 2;
X¹ is O;
X² is C₁-C₆ alkyl or C₁-C₆ alkylamino; and
R², R³, R⁴, R⁵, R⁶, R⁷, R⁸ and R⁹ are independently hydrogen.

11. A compound selected from the group consisting of: 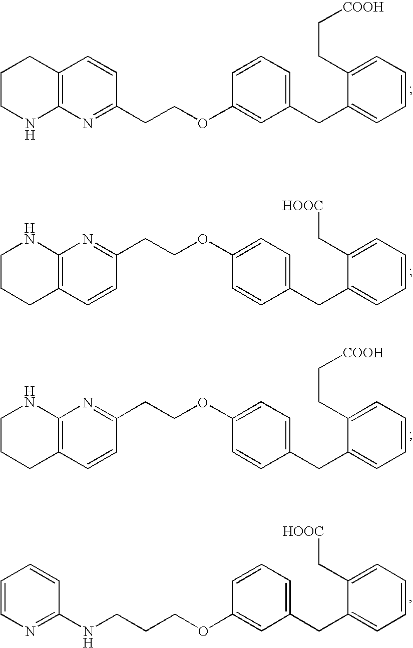
and a pharmaceutically-acceptable salt of the compound or ester of the compound.

12. A pharmaceutical composition comprising a compound of claim 1 and at least one pharmaceutically-acceptable carrier.

13. A method of inhibiting the αᵥβ₃ integrin, the method comprising administering to a patient in need of αᵥβ₃ inhibition, an αᵥβ₃-inhibiting amount of a compound of claim 1.

14. A method of treating an αᵥβ₃ integrin-mediated condition selected from the group consisting of osteoporosis, retinopathy, tumor metastasis and angiogenesis, the method comprising administering to a patient in need thereof a therapeutically-effective amount of a compound of claim 1.
